(19) United States
(12) Patent Application Publication
(10) Pub. No.: US 2022/0117912 A1
(43) Pub. Date: Apr. 21, 2022

(54) L-PIPECOLIC ACID COCRYSTAL OF CANNABIDIOL

(71) Applicant: EBERS TECH INC., Toronto (CA)

(72) Inventors: David T. JONAITIS, Brookston, IN (US); Nathan SCHULTHEISS, West Lafayette, IN (US)

(21) Appl. No.: 17/430,447

(22) PCT Filed: Feb. 14, 2020
(86) PCT No.: PCT/US2020/018224

§ 371 Date: Aug. 12, 2021

Related U.S. Application Data
(60) Provisional application No. 62/806,318, filed on Feb. 15, 2019.

(51) Int. Cl. A61K 31/05; A61K 47/54

(52) U.S. Cl. CPC A61K 31/05; A61K 47/545

(57) ABSTRACT

A cocrystal of cannabidiol is disclosed, specifically a 1:1 cannabidiokL-pipecolic acid cocrystal The beneficial and therapeutic uses of the cocrystal and of compositions containing the cocrystal are also disclosed. The disclosure sets out methods of making and characterizing the cocrystal.

### CROSS-REFERENCE TO RELATED APPLICATIONS

This application claims priority to U.S. application No. 62/806,318 filed Feb. 15, 2019 which is incorporated herein by reference.

### FIELD OF THE INVENTION

The invention relates to a cocrystal of cannabidiol is disclosed, specifically a 1:1 cannabidiol:L-pipecolic acid cocrystal. The invention also relates to beneficial and therapeutic uses of the cocrystal and of compositions containing the cocrystal.

### BACKGROUND OF THE INVENTION

Crystalline forms of therapeutic drugs have been used to alter the physicochemical properties of the drug. Each crystalline form of a drug can have different solid-state (physical and chemical) properties which may be relevant for drug delivery. Crystalline forms often have better chemical and physical properties than corresponding non-crystalline forms such as the amorphous form. The differences in physical properties exhibited by a novel solid form of a drug (such as a cocrystal or polymorph of the original drug) affect pharmaceutical parameters such as melting point, storage stability, compressibility and density (relevant for formulation and product manufacturing), and dissolution rates and solubility (relevant factors in achieving suitable bioavailability).

Obtaining a suitable crystalline form of a drug is often a necessary stage for many orally available drugs. Suitable crystalline forms possess the desired properties of a particular drug. Such suitable crystalline forms may be obtained by forming a cocrystal between the drug and a coformer. Cocrystals often possess more favorable pharmaceutical and pharmacological properties or may be easier to process than known forms of the drug itself. As well, a cocrystal is one way to avoid polymorph formation of the drug. For example, a cocrystal may have different dissolution and solubility properties than the drug. Further, cocrystals may be used as a convenient vehicle for drug delivery, and new drug formulations comprising cocrystals of a given drug may have superior properties. Such as melting point, solubility, dissolution rate, hygroscopicity and storage stability over existing formulations of the drug.

A cocrystal of a drug (a supplement ingredient or an active pharmaceutical ingredient) is a distinct chemical composition between the drug and coformer, and generally possesses distinct crystallographic and spectroscopic properties when compared to those of the drug and coformer individually. Unlike salts, which possess a neutral net charge, but which are comprised of charge-balanced components, cocrystals are comprised of neutral species. Thus, unlike a salt, one cannot determine the stoichiometry of a cocrystal based on charge balance. Indeed, one can often obtain cocrystals having stoichiometric ratios of drug to coformer of greater than or less than 1:1. The stoichiometric ratio of an API to coformer is a generally unpredictable feature of a cocrystal.

Without limiting the present invention to any particular definition because others may define the term differently, the term “cocrystal” may be thought of as a multi-component crystal composed of neutral molecules. These multi-component assemblies are continuing to excite and find usefulness, particularly within the pharmaceutical arena, for their ability to alter physicochemical properties. More specifically, cocrystals have been reported to alter melting point, aqueous solubility and/or dissolution rates, increase stability with respect to relative humidity, and improve bioavailability of active pharmaceutical ingredients.

A necessary consideration when designing cocrystals, if the end goal is a potential marketed drug-product, is incorporating a suitable cocrystal former (coformer) with an acceptable toxicity profile. Within the pharmaceutical industry, coformers are typically selected from the same list of pharmaceutically accepted salt formers, generally regarded as safe (GRAS) and/or everything added to food in the United States (EAFUS) lists, due to previous occurrence of these molecules in FDA approved drug or food products. Utilizing naturally occurring compounds as coformers gives extension to the list of potential molecules accessible to the pharmaceutical industry and provides additional physiological benefits to the consumer.

In a cocrystal, the drug and the coformers each possess unique lattice positions within the unit cell of the crystal lattice. Crystallographic and spectroscopic properties of cocrystals can be analyzed as with other crystalline forms such as with X-ray powder diffraction (XRPD) among other techniques. Cocrystals often also exhibit distinct thermal behavior compared with other forms of the corresponding drug. Thermal behavior may be analyzed by such techniques as capillary melting point, thermogravimetric analysis (TGA), and differential scanning calorimetry (DSC) to name a few. These techniques can be used to identify and characterize the cocrystals.

Cannabidiol (CBD) is one of the greater than 100 cannnabinoids found in the Cannabis sativa plant. See Oier Aizpurua-Olaizola, et al.; Evolution of the Cannabinoid and Terpene Content during the Growth of Cannabis sativa Plants from Different Chemotypes; J. Nat. Prod., 2016, 79 (2), pp 324-331. CBD lacks the psychoactive effects such as those found in tetrahydrocannabinol (THC). As a therapeutic drug, CBD may be suitable for the treatment of diseases or disorders, or symptoms of diseases or disorders, such as Dravet Syndrome, Lennox Gastaut Syndrome, mycolonic seizures, juvenile mycolonic epilepsy, refractory epilepsy, schizophrenia, juvenile spasms, West syndrome, refractory infantile spasms, infantile spasms, tuberous sclerosis complex, brain tumors, neuropathic pain, Cannabis use disorder, post-traumatic stress disorder, anxiety, early psychosis, Alzheimer's Disease, autism, and withdrawal from opioids, cocaine, heroin, amphetamines, and nicotine.

One mention of an unidentified cocrystal of cannabidiol has been reported by Artelo Bio as early July 2018, (see also, e.g., http://artelobio.com/wp-content/uploads/2019/01/Artelo-Bio-Corp-Pres-23JAN2019.pdf). In fact, the field of cannabidiol cocrystals appears to be a relatively unexplored landscape. Artelo Bio in its published US patent application US2019/0177258A1 (which corresponds to published PCT application WO 2019/118360, both incorporated herein by reference) describes therapeutic uses of cannabidiol and discloses four cannabidiol (CBD) cocrystals: cannabidiol L-proline Form A, cannabidiol D-proline Form A, cannabidiol tetramethylpyrazine Material A, and cannabidiol 4,4′-dipyridyl Material A. Artelo Bio conducted a cocrystal screen which included 68 experiments targeting cannabidiol with 34 coformers. The experiments were set up with 1:1, 2:1 or 1:2 API:coformer ratios with additional experiments performed using an excess amount of coformer. The experiments were conducted using a variety of crystallization techniques including cooling, evaporation, slurrying, and solvent-assisted grinding. The majority of those experiments resulted in cannabidiol, coformer, physical mixtures of cannabidiol and coformer, gels, oils, or discolored solutions. The 68 experiments resulted in only the four cannabidiol cocrystals listed above. See US 2019/0177258 A1, paras. [0133]-[0135]. The coformers which failed to result in any cocrystals are not identified. There remains a need, therefore, for other cannabidiol cocrystals.

### SUMMARY OF THE INVENTION

The invention relates to the a 1:1 cannabidiol:L-pipecolic acid cocrystal, 1. The molecular structures of cannabidiol and L-pipecolic acid are shown below

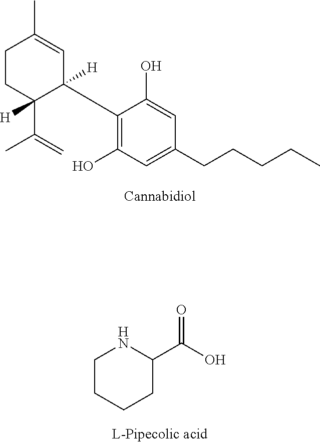 [SMILES: C=C(C)[C@@H]1CCC(C)=C[C@H]1c1c(O)cc(CCCCC)cc1O ; O=C(O)C1CCCCN1]

A 1:1 cannabidiol:L-pipecolic acid cocrystal of the invention may be characterized by: a powder X-ray diffraction pattern having at least three peaks selected from 4.3, 6.8, 10.9, 11.7, 12.8, 21.8 and 22.2° 2θ±0.2° 2θ; a powder X-ray diffraction pattern substantially as shown in FIG. 2; a powder X-ray diffraction pattern substantially as shown in FIG. 8; a DSC trace substantially as shown in FIG. 3; a melting point near 140° C.; or a TGA trace substantially as shown in FIG. 4.

The invention also relates to a composition comprising a 1:1 cannabidiol:L-pipecolic acid cocrystal and a carrier. A particular composition is a pharmaceutical composition comprising a therapeutically effective amount of a 1:1 cannabidiol:L-pipecolic acid cocrystal of the invention and a pharmaceutically acceptable carrier.

The invention further relates to a method of treating a disease, disorder or condition using cannabidiol the improvement comprising administering to a patient in need thereof a beneficial or therapeutically effective amount of a 1:1 cannabidiol:L-pipecolic acid cocrystal, a composition or a pharmaceutical composition of the invention. Similarly the invention relates to the use of cannabidiol to treat a disease, disorder or condition characterized by administering to a patient in need thereof a beneficial or therapeutically effective amount of a 1:1 cannabidiol:L-pipecolic acid cocrystal, a composition or a pharmaceutical composition of the invention.

### BRIEF DESCRIPTION OF THE FIGURES

FIG. 1 shows stacked XRPD patterns of cannabidiol (as-received, top), a 1:1 cannabidiol:L-pipecolic acid cocrystal (middle), and L-pipecolic acid (as-received, bottom).

FIG. 2 is the experimental XRPD Pattern of a 1:1 cannabidiol:L-pipecolic acid cocrystal from Example 1.

FIG. 3 is a DSC trace of a 1:1 cannabidiol:L-pipecolic acid cocrystal.

FIG. 4 is a TGA trace of a 1:1 cannabidiol:L-pipecolic acid cocrystal.

FIG. 5 is the solution ¹H NMR spectrum of a 1:1 cannabidiol:L-pipecolic acid cocrystal.

FIG. 6 is a DSC overlay showing the melting point profile of cannabidiol (as-received, left trace) and a 1:1 cannabidiol:L-pipecolic acid cocrystal (right trace).

FIG. 7 is the XRPD pattern of the 1:1 cannabidiol:L-pipecolic acid cocrystal sample in Example 2.

FIG. 8 is the high-resolution XRPD pattern of the 1:1 cannabidiol:L-pipecolic acid cocrystal in Example 2.

FIG. 9 is a stack plot of a 1:1 cannabidiol:L-pipecolic acid cocrystal sample before and after exposure to 95% relative humidity (RH)—traces from top to bottom: post 95% RH stress, CBD Form A, 1:1 cannabidiol:L-pipecolic acid cocrystal, and L-pipecolic acid.

FIG. 10 is the XRPD pattern of the 1:1 cannabidiol:L-pipecolic acid cocrystal sample post 95% RH stress for 8 days.

### DETAILED DESCRIPTION OF THE INVENTION

The invention relates to a cannabidiol:L-pipecolic acid cocrystal. The cocrystal has a 1:1 stoichiometric molar ratio of cannabidiol with L-pipecolic acid. The 1:1 cannabidiol:L-pipecolic acid cocrystal was prepared and characterized by XRPD, TGA, DSC, and ¹H NMR as described in the examples below. The melting point of cocrystal is 131.18° C. compared to that of pure, crystalline CBD, 67.71° C. and the melting point of L-pipecolic acid, 264° C.

Therapeutic Uses of the Cannabidiol:L-Pipecolic Acid Cocrystal

Cannabidiol (CBD), a phytocannabinoid, is known to be beneficial for human and animal health. The invention also provides therapeutic and beneficial uses of the 1:1 cannabidiol:L-pipecolic acid cocrystal and methods for its delivery, and compositions, such as pharmaceutical dosage forms, containing the cocrystal, to humans. The 1:1 cannabidiol:L-pipecolic acid cocrystal of the invention may then be used, in the same way as cannabidiol, to treat diseases, disorders and conditions, such as those discussed above, and to provide beneficial treatment for humans and animals as with CBD. The invention therefore provides a method of treating a disease, disorder or condition using cannabidiol the improvement comprising administering to a patient in need thereof a beneficial or therapeutically effective amount of a 1:1 cannabidiol:L-pipecolic acid cocrystal, a composition or a pharmaceutical composition of the invention. Similarly the invention relates to the use of cannabidiol to treat a disease, disorder or condition characterized by administering to a patient in need thereof a beneficial or therapeutically effective amount of a 1:1 cannabidiol:L-pipecolic acid cocrystal, a composition or a pharmaceutical composition of the invention.

The invention then also relates to the method of treating (or the use of a 1:1 cannabidiol:L-pipecolic acid cocrystal to treat) such a disease, disorder, or condition by administering to a human or animal patient in need thereof a therapeutically effective or beneficial amount of the cannabidiol:L-pipecolic acid cocrystal of the invention or of administering to a human or animal patient in need thereof a therapeutic composition containing the 1:1 cannabidiol:L-pipecolic acid cocrystal of the invention. A 1:1 cannabidiol:L-pipecolic acid cocrystal of the invention may be used to treat diseases, disorders or conditions in the same way as CBD. This includes, for example, the treatment of diseases, disorders or conditions, and treatment symptoms of diseases, disorders or conditions, such as Dravet Syndrome, Lennox Gastaut Syndrome, mycolonic seizures, juvenile mycolonic epilepsy, refractory epilepsy, schizophrenia, juvenile spasms, West syndrome, refractory infantile spasms, infantile spasms, tuberous sclerosis complex, brain tumors, neuropathic pain, Cannabis use disorder, post-traumatic stress disorder, anxiety, early psychosis, Alzheimer's Disease, autism, and withdrawal from opioids, cocaine, heroin, amphetamines, and nicotine.

The term “treatment” or “treating” means any treatment of a disease, disorder or condition in a mammal, including: preventing or protecting against the disease, disorder or condition, that is, causing the clinical symptoms not to develop; inhibiting the disease, disorder or condition, that is arresting or suppressing, the development of clinical symptoms; and/or relieving the disease, disorder or condition (including the relief of discomfort associated with the condition or disorder), that is, causing the regression of clinical symptoms. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” the disease, disorder or condition. The term “protection” is meant to include “prophylaxis.”

The 1:1 cannabidiol:L-pipecolic acid cocrystal of the invention may be administered at cannabidiol dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired effect. It will also be appreciated that, where appropriate, dosages smaller than 0.001 mg/kg or greater than 50 mg/kg (for example 50-100 mg/kg) can be administered to a subject in need thereof.

Compositions Containing the 1:1 Cannabidiol:L-Pipecolic Acid Cocrystal

The invention also relates to compositions, such as dietary supplement and pharmaceutical compositions, comprising a beneficial or therapeutically effective amount of the 1:1 cannabidiol:L-pipecolic acid cocrystal according to the invention and a carrier, such as a pharmaceutically acceptable carrier (also known as a pharmaceutically acceptable excipient). The compositions and pharmaceutical dosage forms may be administered using any amount, any form of composition, dietary supplement or pharmaceutical composition and any route of administration a beneficial or therapeutically effective for treatment. As mentioned above, these pharmaceutical compositions are therapeutically useful to treat or prevent disorders such as those discussed above.

A pharmaceutical composition of the invention may be in any pharmaceutical dosage form known in the art which contains the 1:1 cannabidiol:L-pipecolic acid cocrystal according to the invention. A composition, particularly a pharmaceutical composition, may be, for example, a tablet, a capsule, a liquid suspension, an injectable composition, a topical composition, an inhalable composition or a transdermal composition. The compositions, particularly pharmaceutical compositions generally contain, for example, about 0.1% to about 99.9% by weight of the 1:1 cannabidiol:L-pipecolic acid cocrystal, for example, about 0.5% to about 99% by weight of the 1:1 cannabidiol:L-pipecolic acid cocrystal of the invention and, for example, 99.5% to 0.5% by weight of at least one suitable carrier such as a pharmaceutically acceptable carrier and/or excipient. The composition may also be between about 5% and about 75% by weight of the 1:1 cannabidiol:L-pipecolic acid cocrystal of the invention with the rest being at least one suitable pharmaceutical carrier and/or excipient, as discussed below.

The dosage form an appropriate pharmaceutically acceptable carrier or excipient in a desired dosage, as known by those of skill in the art, the pharmaceutical compositions of this disclosure can be administered to humans and other animals orally, rectally, parenterally, intravenously, intracisternally, intravaginally, intraperitoneally, topically (as by powders, ointments, or drops), buccally, as an oral or nasal spray, or the like, depending on the location and severity of the condition being treated. In one embodiment, the pharmaceutical composition is with an oral unit dosage form.

Compositions, particularly pharmaceutical compositions, of the invention include a beneficial or therapeutically effective amount of the 1:1 cannabidiol:L-pipecolic acid cocrystal of the invention and a carrier such as a pharmaceutically acceptable carrier and/or excipient. Such pharmaceutically acceptable carriers and excipients, including, without limitation, binders, fillers, lubricants, emulsifiers, suspending agents, sweeteners, flavorings, preservatives, buffers, wetting agents, disintegrants, effervescent agents, and other conventional excipients and additives. The pharmaceutical compositions of the invention can thus include any one or a combination of the following: a pharmaceutically acceptable carrier or excipient; other medicinal agent(s); pharmaceutical agent(s); adjuvants; buffers; preservatives; diluents; and various other pharmaceutical additives and agents known in the art. These additional formulation additives and agents will often be biologically inactive and can be administered to humans without causing deleterious side effects or interactions.

Suitable additives may include, but are not limited to, microcrystalline cellulose, lactose, sucrose, fructose, glucose, dextrose, other sugars, di-basic calcium phosphate, calcium sulfate, cellulose, methylcellulose, cellulose derivatives, kaolin, mannitol, lactitol, maltitol, xylitol, sorbitol, other sugar alcohols, dry starch, dextrin, maltodextrin, other polysaccharides, or mixtures thereof.

In one embodiment of the invention the pharmaceutical composition is an oral unit dosage form containing a therapeutically effective amount of the 1:1 cannabidiol:L-pipecolic acid cocrystal of the invention and a pharmaceutically acceptable carrier and/or excipient. Exemplary oral unit dosage forms for use in the present disclosure include tablets, capsules, powders, suspensions, and lozenges, which may be prepared by any conventional method of preparing pharmaceutical oral dosage forms. Oral unit dosage forms, such as tablets, may contain one or more pharmaceutically acceptable carriers and/or excipients such as known in the art as discussed above, including but not limited to, release modifying agents, glidants, compression aides, disintegrants, effervescent agents, lubricants, binders, diluents, flavors, flavor enhancers, sweeteners, and preservatives.

Tablet dosage forms may be partially or fully coated, sub-coated, uncoated, and may include channeling agents. The ingredients are selected from a wide variety of excipients known in the pharmaceutical formulation art. Depending on the desired properties of the oral dosage form, any number of ingredients may be selected alone or in combination for their known use in preparing such dosage forms as tablets.

### EXAMPLES

The following reagents and analytical methods were used to prepare and characterize the 1:1 cannabidiol:L-pipecolic acid cocrystal of the invention.

Reagents: Cannabidiol was acquired from Cayman Chemical and used as received. L-Pipecolic acid was purchased from Sigma-Aldrich and used as received. All other chemicals were purchased from various suppliers and used without further purification.

X-Ray Powder Diffraction (XRPD): The Rigaku Smart-Lab X-ray diffraction system was configured for reflection Bragg-Brentano geometry using a line source X-ray beam. The x-ray source is a Cu Long Fine Focus tube that was operated at 40 kV and 44 ma. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0-degree Soller slits in both the incident and diffracted beam paths. Powder samples were prepared in a low background Si holder using light manual pressure to keep the sample surfaces flat and level with the reference surface of the sample holder. Each sample was analyzed from 2 to 40° 2θ using a continuous scan of 6° 2θ per minute with an effective step size of 0.02° 2θ. In the case of high resolution XRPD, samples were analyzed with a scan rate of 1° 2θ per minute with a sample spin rate of 50 rpm.

Differential Scanning calorimetry (DSC): DSC analyses were carried out using a TA Instruments Q2000 instrument. The instrument temperature calibration was performed using indium. The DSC cell was kept under a nitrogen purge of ^{˜}50 mL per minute during each analysis. The sample was placed in a standard, crimped, aluminum pan and was heated from 25° C. to 350° C. at a rate of 10° C. per minute.

Thermogravimetric Analysis (TGA): The TG analysis was carried out using a TA Instruments Q50 instrument. The instrument balance was calibrated using class M weights and the temperature calibration was performed using alumel. The nitrogen purge was ^{˜}40 mL per minute at the balance and ^{˜}60 mL per minute at the furnace. Each sample was placed into a pre-tared platinum pan and heated from 20° C. to 350° C. at a rate of 10° C. per minute.

Solution ¹H Nuclear Magnetic Resonance (NMR) Spectroscopy: The ¹H NMR spectra were acquired on a Bruker DRX-500 spectrometer located at the Chemistry Department of Purdue University. Samples were prepared by dissolving material in DMSO-d₆. The solutions were filtered and placed into individual 5-mm NMR tubes for subsequent spectral acquisition. The temperature controlled (298K)¹H NMR spectra acquired on the DRX-500 utilized a 5-mm cryoprobe operating at an observing frequency of 499.89 MHz.

### Example 1: Preparation and Characterization of the 1:1 Cannabidiol:L-Pipecolic Acid Cocrystal

Cannabidiol (27.1 mg) and L-pipecolic acid (11.3 mg) were weighed into a 2-dram glass vial. Approximately, 1 mL of absolute ethanol was added, sample sonicated, and solids remained. Approximately, 1 mL of methanol was added to the vial, sample sonicated, and all the solids dissolved. A dry air purge was run into the vial, resulting in a clear film. The vial was then placed in a room temperature vacuum desiccator overnight. White solids were observed the next day.

1.1: XRPD Characterization of the 1:1 Cannabidiol:L-Pipecolic Acid Cocrystal

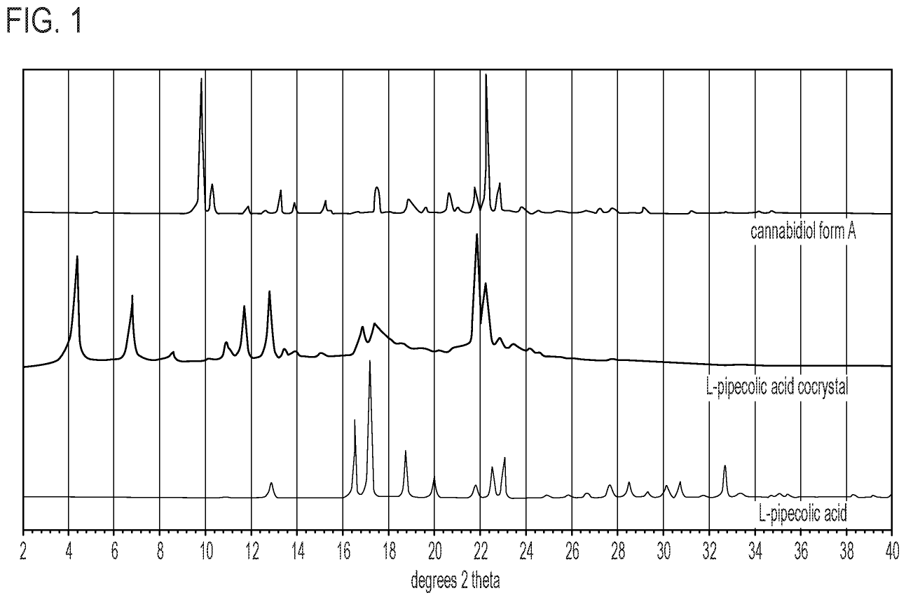

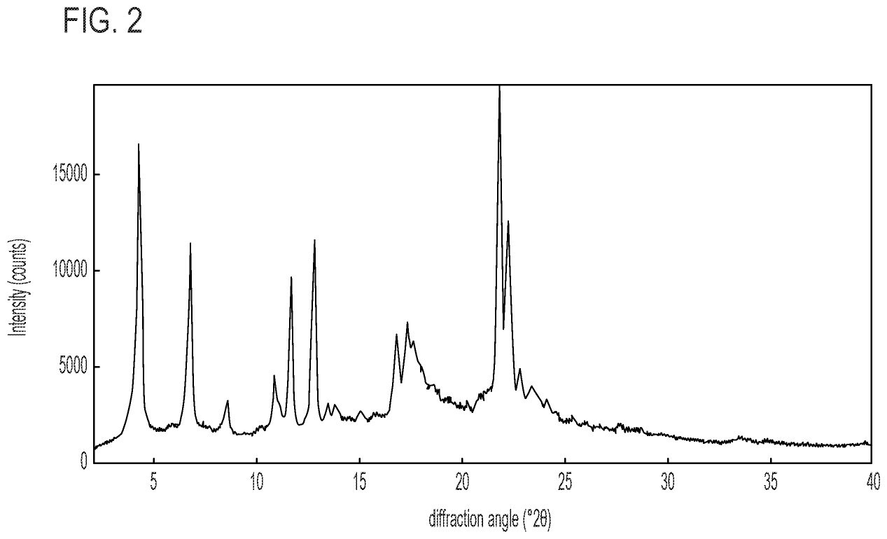

The stacked XRPD patterns of cannabidiol (as-received, top), 1:1 cannabidiol:L-pipecolic acid cocrystal (middle), L-pipecolic acid (bottom), are shown in FIG. 1. The experimental XRPD pattern of the 1:1 cannabidiol:L-pipecolic acid cocrystal is shown in FIG. 2. Table 1 lists the angles, ° 2θ±0.2° 2θ, and d-spacing of the peaks identified in the experimental XRPD pattern of FIG. 2. The entire list of peaks, or a subset thereof, may be sufficient to characterize the cocrystal, as well as by an XRPD pattern substantially similar (that is, identifiable by one of ordinary skill using a characterization method, such as this one, within experimental variations) to FIG. 2. For example, a 1:1 cannabidiol:L-pipecolic acid cocrystal of the invention may be characterized by a powder X-ray diffraction pattern having at least three peaks selected from 4.3, 6.8, 10.9, 11.7, 12.8, 21.8 and 22.2° 2θ±0.2° 2θ.

TABLE 1

| 2-theta (deg) | d-spacing (Å) | Relative Intensity (I) |
|---|---|---|
| 4.3 | 20.47 | 100 |
| 6.8 | 13.07 | 52.9 |
| 8.6 | 10.33 | 9.0 |
| 10.9 | 8.14 | 18.1 |
| 11.7 | 7.58 | 38.2 |
| 12.8 | 6.92 | 50.4 |
| 13.5 | 6.57 | 4.0 |
| 13.9 | 6.38 | 5.5 |
| 15.0 | 5.89 | 1.2 |
| 16.8 | 5.27 | 22.5 |
| 17.3 | 5.12 | 14.8 |
| 17.7 | 5.02 | 34.1 |
| 18.1 | 4.91 | 7.8 |
| 18.5 | 4.80 | 17.0 |
| 21.8 | 4.08 | 78.7 |
| 22.2 | 4.00 | 57.9 |
| 22.8 | 3.89 | 11.8 |
| 23.4 | 3.80 | 12.4 |
| 24.1 | 3.69 | 3.4 |
| 28.6 | 3.12 | 10.1 |
| 33.5 | 2.67 | 5.0 |

1.2: DSC of the 1:1 Cannabidiol:L-Pipecolic Acid Cocrystal

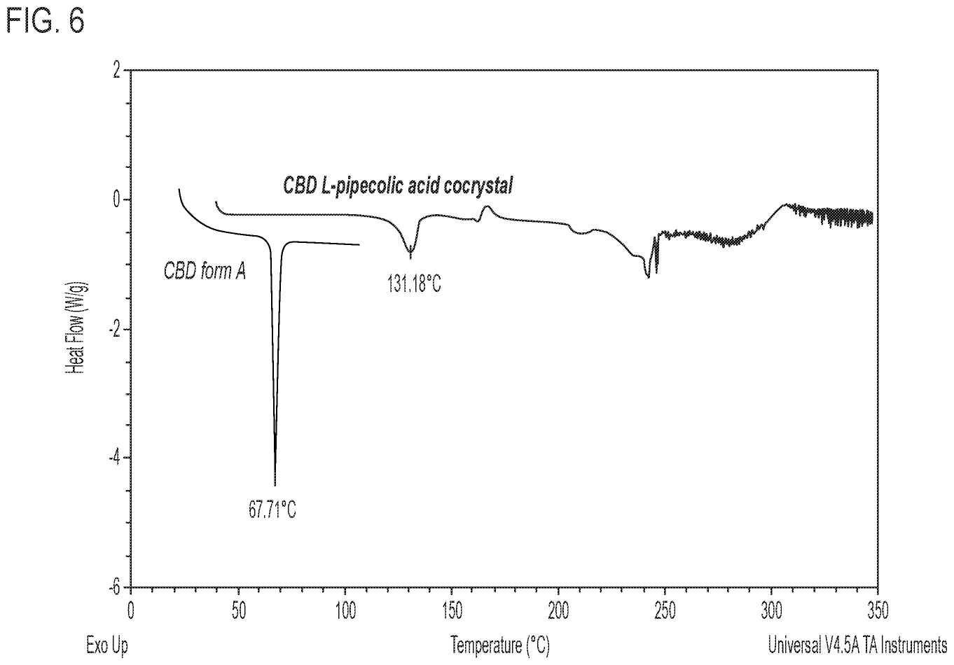

The differential scanning calorimetry (DSC) trace, FIG. 6, shows a single endotherm with a peak maximum of 131.18° C. corresponding to the melt of the cocrystal. For cannabidiol form a single endotherm with a peak maximum of 67.71° C. is observed. The sample size was 1.69 mg. FIG. 6 is a DSC overlay showing the melting point profile of cannabidiol (as-received, left trace) and a 1:1 cannabidiol:L-pipecolic acid cocrystal (right trace).

1.3: TGA of the 1:1 Cannabidiol:L-Pipecolic Acid Cocrystal

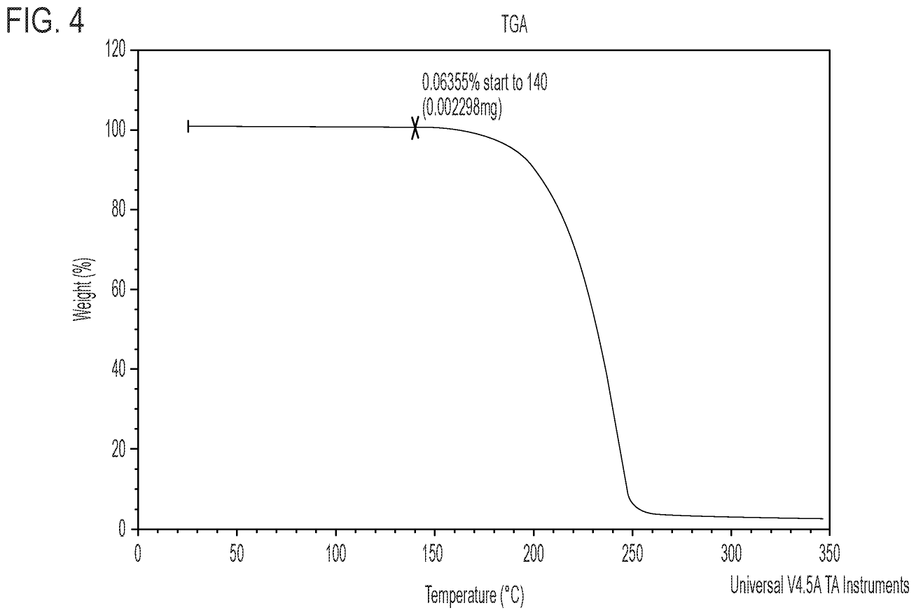

The thermal gravimetric analysis (TGA) trace, FIG. 4, shows no significant weight loss prior to the cocrystal melt temperature near 140° C. The sample size was 3.62 mg.

¹H NMR Spectrum of the 1:1 Cannabidiol:L-Pipecolic Acid Cocrystal

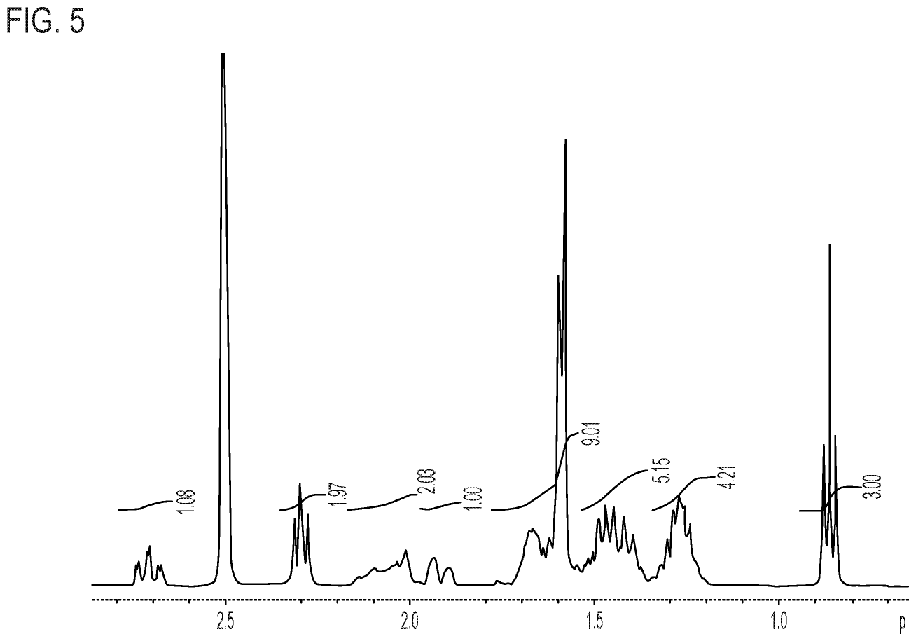

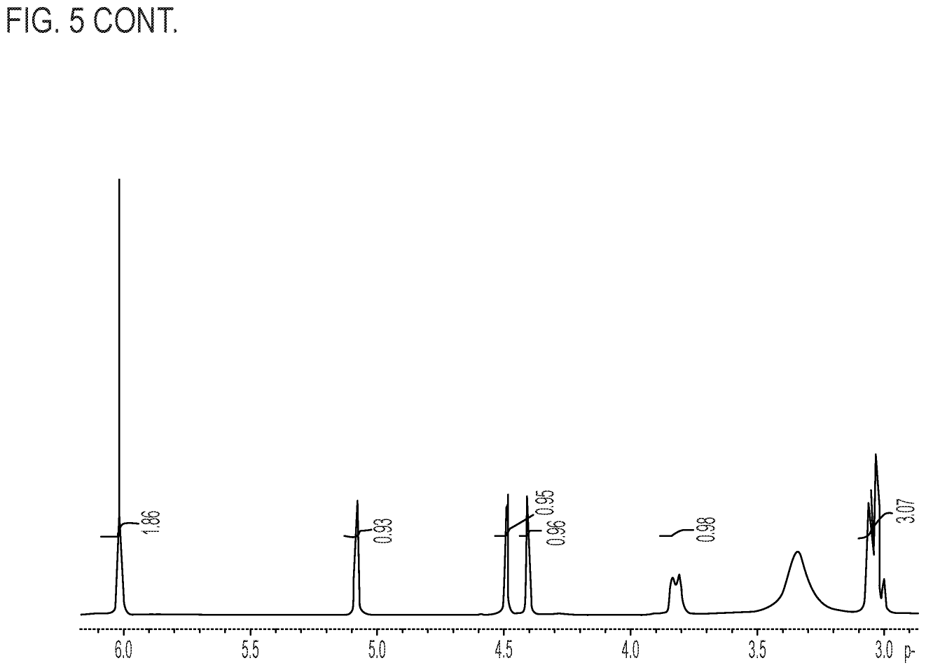

The ¹H NMR spectrum of the 1:1 cannabidiol L-pipecolic acid shown in FIG. 5, displays the following peaks: ¹H NMR (400 MHz, d6-DMSO) δ: 6.02 (2H), 5.08 (1H), 4.49 (1H), 4.41 (1H), 3.82 (1H), 3.03 (3H), 2.71 (1H), 2.30 (2H), 2.03 (2H), 1.91 (1H), 1.68-1.38 (19H), 1.45 (2H), 1.27 (4H), 0.86 (3H). The peak at 6.02 ppm in the ¹H NMR spectrum corresponds to two protons on the aromatic ring of cannabidiol. Comparison of the integration of this peak with the multiplet at 2.71 ppm, which corresponds to one of the protons of L-pipecolic acid indicates that the cocrystal has a cannabidiol:L-pipecolic acid stoichiometry of 1:1.

### Example 2: Preparation and Characterization of the 1:1 Cannabidiol:L-Pipecolic Acid Cocrystal

Cannabidiol (32.0 mg) and L-pipecolic acid (13.2 mg) were combined in a 2-dram glass vial. Approximately, 2 mL of 1:1 methanol:ethanol was added and the sample was sonicated to dissolve. An air purge was run into the vial, resulting in a clear solid residue on the vial walls after 1 day. The solids were scraped off the sides of the vial and were placed in a room temperature vacuum desiccator for 1 day.

2.1: XRPD Characterization of the 1:1 Cannabidiol:L-Pipecolic Acid Cocrystal

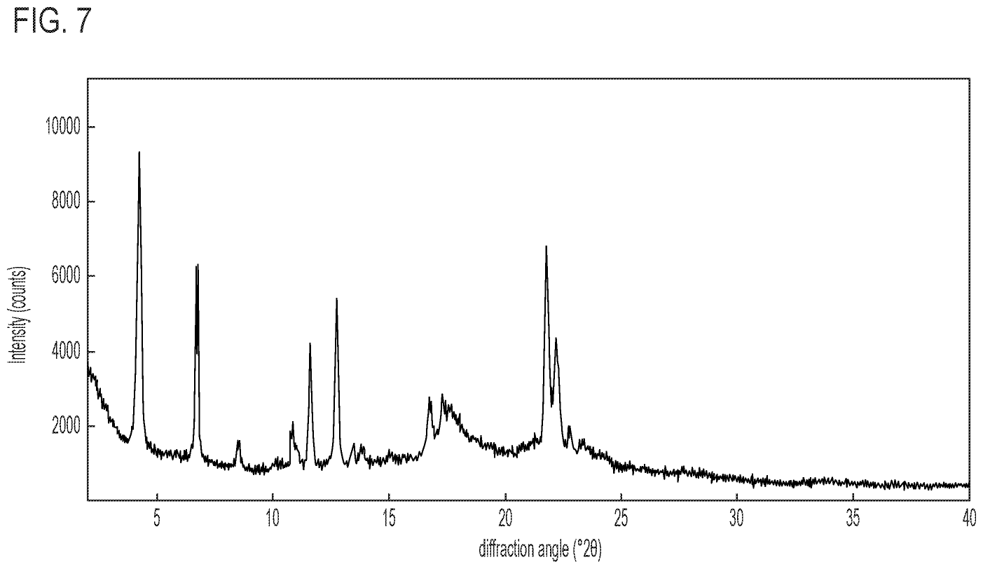

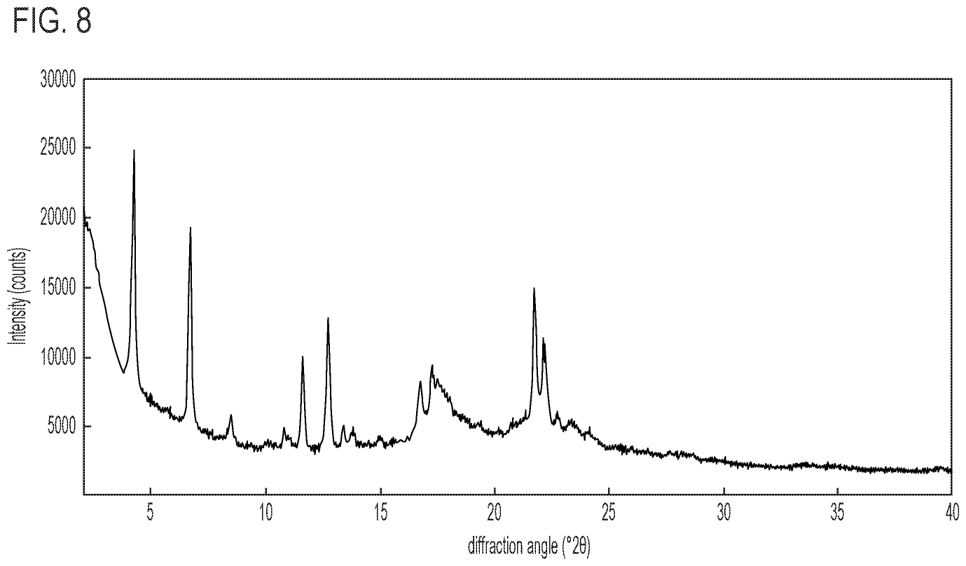

The L-pipecolic acid cocrystal of cannabidiol was analyzed by XRPD. The XRPD pattern for the prepared 1:1 cannabidiol:L-pipecolic acid cocrystal sample is shown in FIG. 7. The high-resolution XRPD pattern for the 1:1 cannabidiol:L-pipecolic acid cocrystal is shown in FIG. 8. Table 2 lists the angles, ° 2θ±0.2° 2θ, and d-spacing of the peaks identified in the experimental XRPD pattern of FIG. 8. The entire list of peaks, or a subset thereof, may be sufficient to characterize the cocrystal, as well as by an XRPD pattern substantially similar to FIG. 7 or FIG. 8. For example, a 1:1 cannabidiol:L-pipecolic acid cocrystal of the invention may be characterized by a powder X-ray diffraction pattern having at least three peaks selected from 4.3, 6.7, 10.8, 11.6, 12.7, 17.3, 21.7 and 22.1° 2θ±0.2° 2θ.

TABLE 2

| 2-theta (deg) | d-spacing (Å) | Relative Intensity (I) |
|---|---|---|
| 4.3 | 20.70 | 90.8 |
| 6.7 | 13.20 | 69.0 |
| 8.5 | 10.43 | 9.3 |
| 10.8 | 8.20 | 16.8 |
| 11.6 | 7.61 | 31.8 |
| 12.7 | 6.96 | 62.1 |
| 13.4 | 6.60 | 6.5 |
| 13.8 | 6.43 | 8.1 |
| 16.8 | 5.28 | 37.9 |
| 17.3 | 5.13 | 100 |
| 17.9 | 4.96 | 48.2 |
| 21.7 | 4.09 | 77.5 |
| 22.1 | 4.01 | 55.5 |
| 23.5 | 3.79 | 74.0 |
| 28.6 | 3.12 | 7.6 |
| 33.3 | 2.61 | 9.7 |

2.2: Visual Characterization of the 1:1 Cannabidiol:L-Pipecolic Cocrystal

0.6 mg of the cocrystal was placed in a glass vial and 3 mL of water was added. The slurry was immediately shaken and sonicated. It appeared that none of the solid dissolved.

2.3 1:1 Cannabidiol:L-Pipecolic Cocrystal Exposure to Relative Humidity (RH) Stress

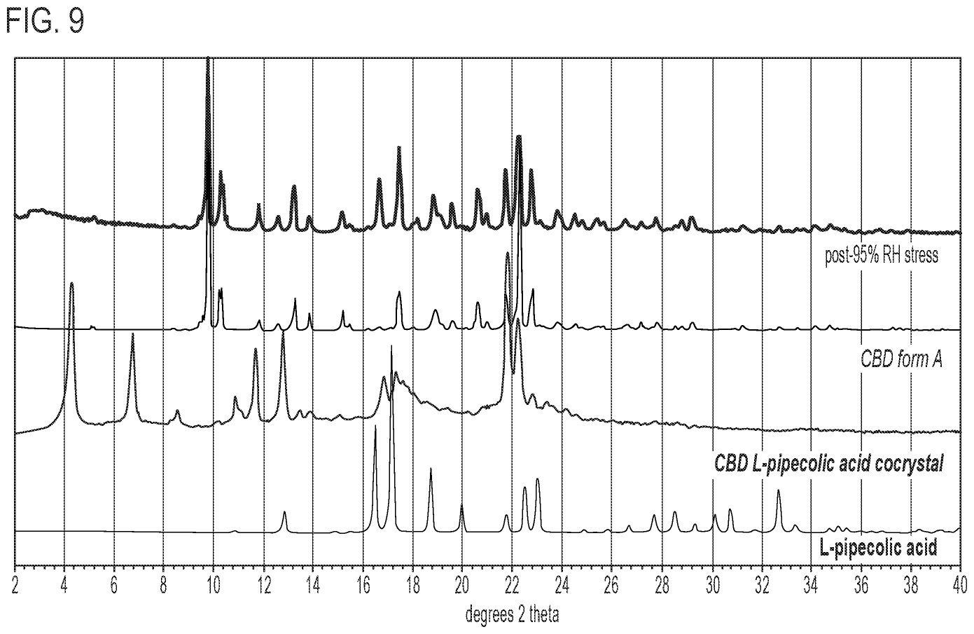

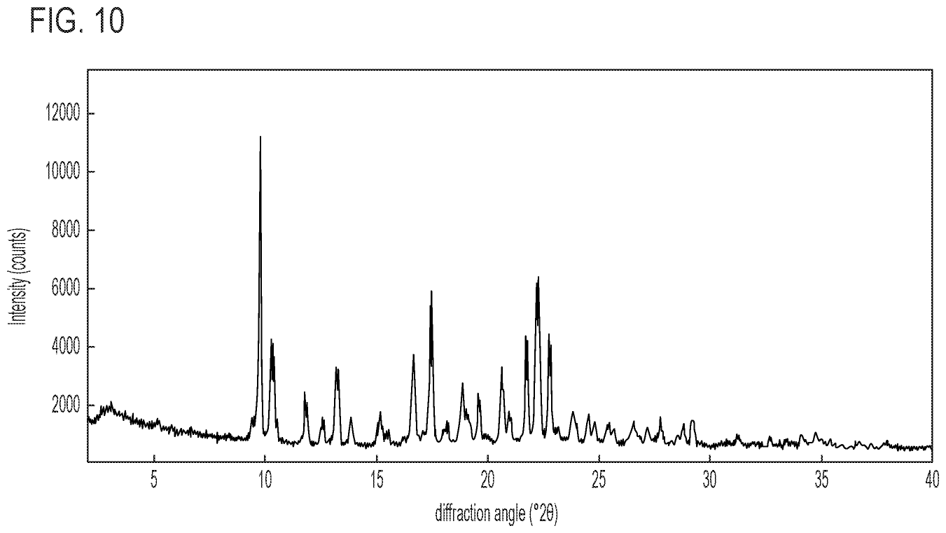

The cocrystal was exposed to 95% RH for 8 days. The resulting material was light brown in color. The XRPD pattern of the material is consistent with free cannabidiol, suggesting that the cocrystal disassociated during the experiment. The L-pipecolic acid likely deliquesced as it is water soluble. The water solubility was less than 0.2 mg/mL. FIG. 9 is a stack plot of a 1:1 cannabidiol:L-pipecolic acid cocrystal sample before and after exposure to 95% relative humidity (RH)—traces from top to bottom: post 95% RH stress, CBD Form A, 1:1 cannabidiol:L-pipecolic acid cocrystal, and L-pipecolic acid. The XRPD pattern of the cocrystal sample post 95% RH stress for 8 days is shown in FIG. 10.

The claimed invention is:

1. A 1:1 Cannabidiol:L-pipecolic acid cocrystal.

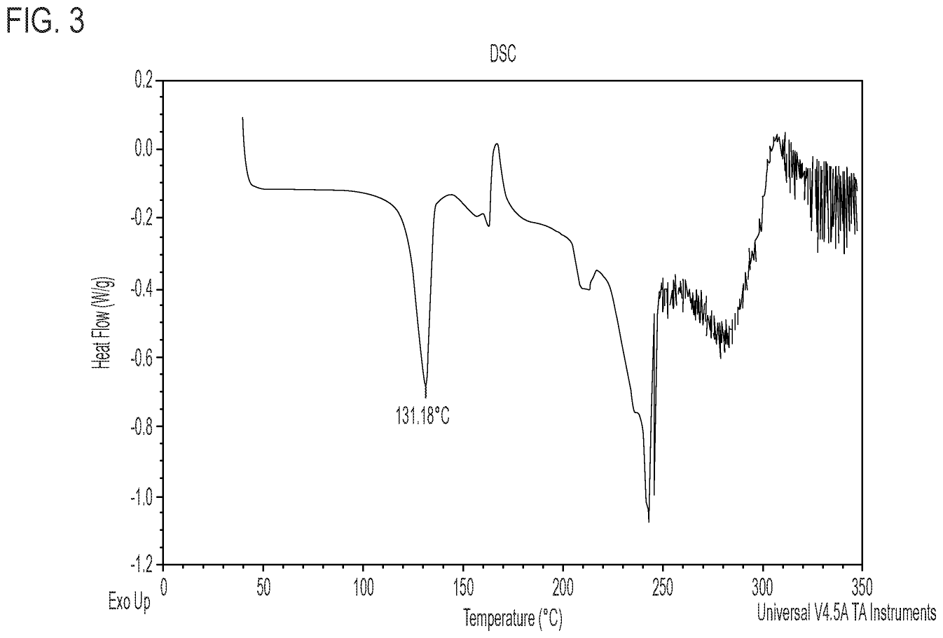

2. The 1:1 Cannabidiol:L-pipecolic acid cocrystal of claim 1 characterized by:
a powder X-ray diffraction pattern having at least three peaks selected from 4.3, 6.8, 10.9, 11.7, 12.8, 17.3, 21.8 and 22.2° 2θ±0.2° 2θ;
a powder X-ray diffraction pattern substantially as shown in FIG. 2;
a powder X-ray diffraction pattern substantially as shown in FIG. 7;
a powder X-ray diffraction pattern substantially as shown in FIG. 8;
a DSC trace substantially as shown in FIG. 3;
a melting point near 140° C.; or
a TGA trace substantially as shown in FIG. 4.

3. A composition comprising a 1:1 cannabidiol:L-pipecolic acid cocrystal of claim 1 or 2 and a carrier.

4. A pharmaceutical composition comprising a therapeutically effective amount of a 1:1 cannabidiol:L-pipecolic acid cocrystal of claim 1 or 2 and a pharmaceutically acceptable carrier.

5. A method of treating a disease, disorder or condition using cannabidiol the improvement comprising administering to a patient in need thereof a beneficial or therapeutically effective amount of a 1:1 cannabidiol:L-pipecolic acid cocrystal of claim 1 or 2, a composition of claim 3 or a pharmaceutical composition of claim 4.

6. The use of cannabidiol to treat a disease, disorder or condition characterized by administering to a patient in need thereof a beneficial or therapeutically effective amount of a 1:1 cannabidiol:L-pipecolic acid cocrystal of claim 1 or 2, a composition of claim 3 or a pharmaceutical composition of claim 4.
